(19) United States
(12) Patent Application Publication
(10) Pub. No.: US 2023/0281346 A1
(43) Pub. Date: Sep. 7, 2023

(54) Customized Foot Support And Systems And Methods For Providing Same

(71) Applicants: United States Government as Represented by the Department of Veterans Affairs, Washington, DC (US); University of Washington, Seattle, WA (US)

(72) Inventors: Patrick Aubin, Seattle, WA (US); Jeffrey Heckman, Seattle, WA (US); George Eli Kaufman, Seattle, WA (US); Daniel Abrahamson, Seattle, WA (US); William Ledoux, Seattle, WA (US); Beth Ripley, Seattle, WA (US); Brittney Muir, Seattle, WA (US); Jing-Sheng Li, Seattle, WA (US); Chris Richburg, Seattle, WA (US); Brian Strzelecki, Seattle, WA (US)

(21) Appl. No.: 18/179,555

(22) Filed: Mar. 7, 2023

Related U.S. Application Data
(60) Provisional application No. 63/317,280, filed on Mar. 7, 2022.

(51) Int. Cl. G06F 30/10; A61F 5/30

(52) U.S. Cl. CPC G06F 30/10; A61F 5/30; G06F 2113/10

(57) ABSTRACT

Insoles and methods for making shoe insoles are disclosed An insole model can be generated based on a profile of a foot and a patient-specific pressure distribution. The profile of the foot can comprise a scan of a crush box impression. An insole can be formed from the insole model.

### CROSS-REFERENCE TO RELATED APPLICATION

This application claims the benefit of the filing date of U.S. Provisional Patent Application No. 63/317,280, filed Mar. 7, 2022, the entirety of which, including the appendices, is hereby incorporated by reference herein.

### STATEMENT OF GOVERNMENT SUPPORT

This invention was made with government support under Department of Veterans Affairs Rehabilitation Research and Development Grant Nos. RX002130, A9243C, and RX002357. The government has certain rights to this invention.

### FIELD

This disclosure relates to apparatuses related to footwear comfort and systems and methods for providing same.

### BACKGROUND

Patients with diabetes mellitus and secondary complications to the peripheral nervous system are at increased risk of developing foot ulcers. In the United States, more than 100,000 patients require amputations each year as a result of diabetic foot wounds that have failed to heal, with 60% of these wounds being foot ulcers. This number is expected to increase due to an aging population. Management of the diabetic foot with the goal of avoiding amputation falls into two main categories: wound prevention and wound healing. Prevention strategies focus on patient education and close monitoring, and the prescription of specialized offloading orthotic devices. If a wound occurs, these two strategies are augmented with the prescription of antibiotic and specialized wound healing treatments. The successes of all of these strategies benefit from specialized care through patient-specific strategies. Foot ulceration in particular is multifactorial and is commonly correlated with elevated plantar pressure levels. Therapeutic offloading devices have been prescribed to reduce plantar pressure and prevent ulceration or promote recovery of existing ulcers. The current standard of care (SoC) includes accommodative shoes and custom insoles designed by specialized clinicians that utilize layers of foam with varying durometers and specially placed additions like heel lifts or metatarsal pads. Patient-specific customized insoles, which rely on clinical expertise and time-consuming manual labor for specific construction materials and shaping techniques, have been shown to be a successful option in reducing and redistributing peak plantar pressures, thereby increasing the ability of the patient to avoid a first ulcer or re-ulceration.

### SUMMARY

Shoe insoles and methods for making shoe insoles are disclosed herein. An insole model can be generated based on a profile of a foot and a patient-specific pressure distribution. The profile of the foot can comprise a scan of a crush box impression. An insole can be formed from the insole model.

Additional advantages of the disclosed apparatuses, systems, and methods will be set forth in part in the description that follows, and in part will be obvious from the description, or may be learned by practice of the claimed invention. The advantages of the disclosed devices and systems will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIGS. 1A-1C illustrate three types of insoles that were fabricated. FIG. 1A illustrates a standard of care (SoC) insole. FIG. 1B illustrates a hybrid 3D printed insole with bilaminate foam top (Hybrid). FIG. 1C illustrates a full 3D printed insole.

FIG. 2 shows heat maps of a representative plantar pressure distribution in a patient with diabetes during walking (from Left to Right): in the standardized footwear (RS), the Standard of care insole (SoC), Hybrid 3D printed insole, and the full 3D printed insole.

FIG. 3 shows a graph of peak plantar pressure during walking across the four insole conditions of FIG. 2. Region of interest (ROI; black), Adjacent region (ADJ; grey). * indicates p-value<0.05. Error bars represent standard error.

FIG. 4 shows a graph of pressure-time integral during walking across the insole conditions of FIG. 2. region of interest (ROI; black), adjacent region (ADJ; grey). * indicates p-value<0.05 for ROI, [symbol] indicates p-value<0.05 for ADJ. Error bars represent standard error.

FIGS. 5A-5D shows different lattice patterns to achieve specific metamaterial properties. Several different lattice properties that can be used to control the macro level metamaterial stiffness. For example: FIG. 5A shows element diameter and element spacing; FIG. 5B shows element arrangement and cross-sectional element geometry; and FIG. 5C shows orientation. FIG. 5D shows a visualization of cubic lattice formation by extrusion through principal axes. FIG. 5E shows a stress-strain comparison of solid material and latticed metamaterials. The solid plug (purple line) exhibits the highest stiffness, but increased lattice element diameter results in decreased stiffness.

FIG. 6A shows a 3D printed sample puck with different lattices. Shown here is a single puck with fixed unit cell size and six different lattice percent infills. The lattice in the top center of sample puck is most sparse (softest) as compared to the six other lattices which increase in density (stiffness) in counterclockwise order. FIG. 6B shows four different lattice designs after months of in shoe beta testing. FIG. 6C shows, after 50,000 steps, this material lattice combination had a mechanical failure and so it was abandoned as a possible solution in preference to the more durable EPU 41 material from Carbon Inc.

FIG. 7A-7C show stress strain curves for the EVA Shore A 50-55, EVA Shore A 35-45, and bilaminate sheet, respectively, as measured from three different samples (orange dotted lines) versus three different Voronoi lattice patterns. The Voronoi lattice metamaterials accurately match the stiffness of the foam bodies up to approximately 50% stain, which is the maximum we expect these materials to strain to.

FIG. 8 shows a flow chart for FE simulation workflow for developing and improving the insoles as disclosed herein.

FIG. 9 shows a block diagram of a computing system for providing an insole model as disclosed herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “an insole” can refer to one or more of such insoles, and so forth.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “substantially” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of” For example, “at least one of A, B and C” explicitly includes only A, only B, only C, and combinations of each.

The word “or” as used herein means any one member of a particular list and, unless context dictates otherwise, can also include any combination of members of that list.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

Additive manufacturing methods, specifically 3-dimensional (3D) printing, can be a viable method for producing custom insoles. One of the most impactful attributes in this space is the ability to programmatically modulate stiffness in three dimensions through modification of the internal geometry of the 3D printed components. A custom 3D model of an insole can be generated using a 3D scan of a foam crush box impression. The insole can then be manufactured using an appropriate polymeric material with internal geometry strategically designed to create regions of varying stiffness throughout the insole. Different 3D printing methods can be used based on the desired properties of custom 3D printed insoles. A fused deposition modeling (FDM) method of 3D printing, in which a plastic filament is extruded through a heated nozzle and deposited in layers to form 3D geometry, can be used to manufacture insoles. The FDM material used in early efforts has typically been limited to common thermoplastic filaments such as polylactic acid (PLA) or acrylonitrile butadiene styrene (ABS). While it is a ubiquitous and low-cost prototyping method, FDM printing technology currently faces some limitations. FDM printing allows the creation of a fully-geometrically-customized insole; however, materials available using this method under-perform as a final product in terms of durability and mechanical property performance. Specifically, when used with stiffer PLA and ABS materials, FDM printing produces rigid, brittle prints and is typically generated only as proof of concept in the initial stages of prototyping.

The characteristics of inter-layer adhesion in FDM printing technology results in prints with largely anisotropic mechanical properties. In some circumstances, prints with largely anisotropic mechanical properties may be desirable. In other circumstances, prints with largely anisotropic mechanical properties may induce early failure in devices fabricated in this manner.

UV-curable resin printing technologies are capable of producing complex print geometries with improved mechanical properties, and have demonstrated the ability to produce isotropic materials. Methods can use parts produced via a UV-cured resin process developed by the Carbon 3D printing technology company (Carbon, Redwood City, Calif.) to characterize the ability to modulate stiffness by lattice geometry design and geometric modification; these elastomers can extend the stiffness modulation beyond the FDM method and show potential in mimicking the material properties of the SoC insoles. The Carbon® Design Engine software allows tuning of lattice geometry and simulation of predicted mechanical properties. Furthermore, the 3D printing process developed by Carbon has demonstrated the ability to produce prints with true isotropic material properties. The actual performance and longevity of elastomers in 3D printed insoles can be evaluated.

Disclosed herein are: (a) methods to produce a 3D printed lattice material that matches the SoC compressive stiffness; (b) methods to develop an insole material with improved durability over the SoC material; (c) methods to reduce the shear stiffness of the insole material over the SoC material; (d) methods to allow the customization of insole stiffness based on patient plantar pressure data; and (e) methods to develop a repeatable workflow for the manufacturing of patient-specific 3D printed plantar pressure-offloading insoles and for the rigorous characterization of new 3D printed metamaterial samples for future insole development. Disclosed herein is a novel 3D printed insole manufacturing workflow. Different fully customized patient-specific insole devices have been developed through the proposed workflow. A first exemplary device is formed as a fully-3D printed material, and a second device was constructed as a hybrid of 3D printed base material and a bi-laminate foam top layer. A testing method was used to determine the durability performance and shear stiffness of insole material samples. An in-vivo case study was performed to demonstrate how the novel 3D printed insole devices applied in a real-life use case in terms of measured plantar pressure during walking.

### Conventional Standard of Care Methods

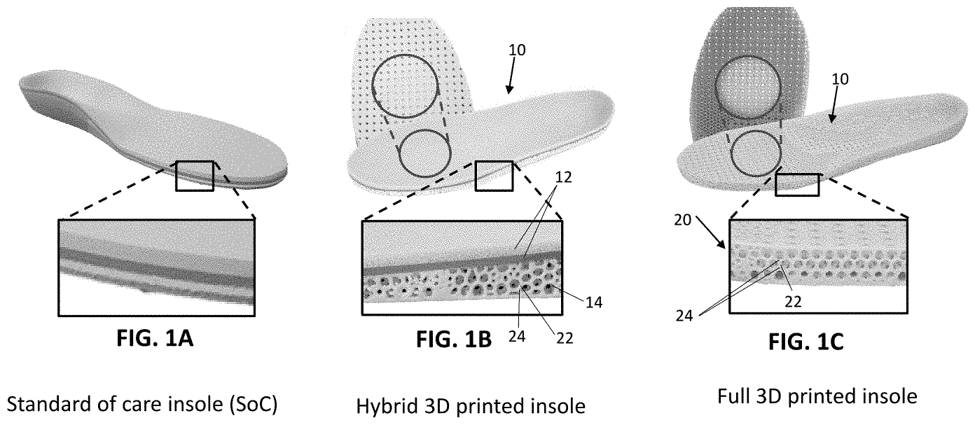

The fabrication of current standard of care (SoC) custom insoles for the treatment of diabetic feet relies heavily on clinical expertise and manual adjustments to accommodate various patient-specific conditions. Typically, a certified orthotist uses a foam crush box to obtain a negative impression of a patient's foot geometry and then fills the crush box with wet plaster, which, once cured, yields a hard positive model of the patient's foot. After a series of manually performed modifications to the plaster model, SoC insoles are constructed by heat- and vacuum-forming layers of foam of different compositions and applying an adhesive between the foam sheets to form a layered foam construction. In this approach, the pressure-relieving region may be considered based on clinical judgments and is produced using a range of strategies that include: 1) the addition of material to the positive plantar model to create a manufactured depression in the finished foot orthosis or 2) by insetting a disc of low-density foam or 3) by removing material from the base of the insole in the desired region or 4) some combination of all these strategies. The insole is then manually shaped to ensure proper fit to the patient's foot and shoes. This entire fabrication process, from generating the foam crush box impression to fitting the insole to the patient and the patient's shoe, typically requires two patient visits over multiple days, sometimes weeks (FIG. 1A).

### Systems and Methods for Providing Custom Insoles

Disclosed herein, in one aspect, is method a method for providing a custom insole. The method comprises generating, based on a profile of a foot and a patient-specific pressure distribution, an insole model. The insole model can be an electronic file representing the geometry of the insole. For example, the insole model can be a 3D computer aided design (CAD) file or any file comprising the information necessary for generating a 3D model or ultimately being printed via a 3D printer. For example, the insole model can comprise an electronic file that is convertible to a file type that a 3D printer can use to form a 3D printed insole.

In some aspects, the profile of the foot can be a 3-dimensional profile. For example, the profile of the foot can comprise a scan of a crush box impression. For example, once a patient steps in a crush box, a laser scanner can capture the 3D geometry of the profile in the crush box. In other aspects, a 3D scan of the foot of the patient can be performed to provide the profile of the foot.

The method can further comprise comprising forming an insole 10 in accordance with the insole model. For example, the insole 10 can be printed based on the insole model. The insole can further be trimmed to any desired shape. In some aspects, one or more additional layers 12 can be added to a 3D printed portion (a base 14) to form the insole 10 (e.g., to provide a hybrid insole as disclosed herein and illustrated in FIG. 1B. In some aspects, the one or more additional layers 12 can be adhered to the 3D printed portion of the insole.

In some aspects, the method can comprise acquiring the patient-specific pressure distribution. For example, the patient-specific pressure distribution can comprise a reading from a pressure sensing pad. In some optional aspects, the pressure sensing pad can be placed within a shoe. The pressure sensing pad can be used to take pressure measurements while the patient is standing and/or while the patient is walking.

In some aspects, generating the insole model can comprise generating a lattice 20.

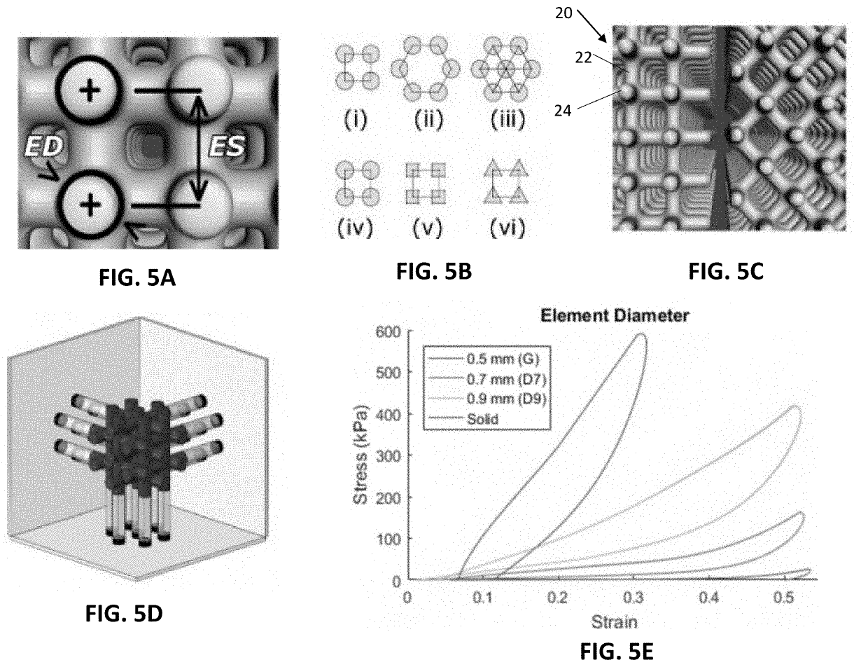

For example, referring to FIGS. 1B, 1C, and 5, the lattice comprises a plurality of elongate segments 22 and a plurality of intersections 24 between elongate segments.

In some aspects, the lattice 20 can comprise at least one repeating pattern. For example, the at least one repeating pattern can comprise hexagonal (e.g., honeycomb) structures. In another aspect, the at least one repeating pattern can comprise triangular structures. For example, the at least one repeating pattern can comprise tetrahedral or pyramidal structures. In another aspect, the at least one repeating pattern can comprise rectancular structures (e.g., rectangular prisms or cubic prisms). In still other aspects, the lattice 20 can comprise at least one irregular shape.

In some aspects, the lattice 20 can provide nonlinear compression.

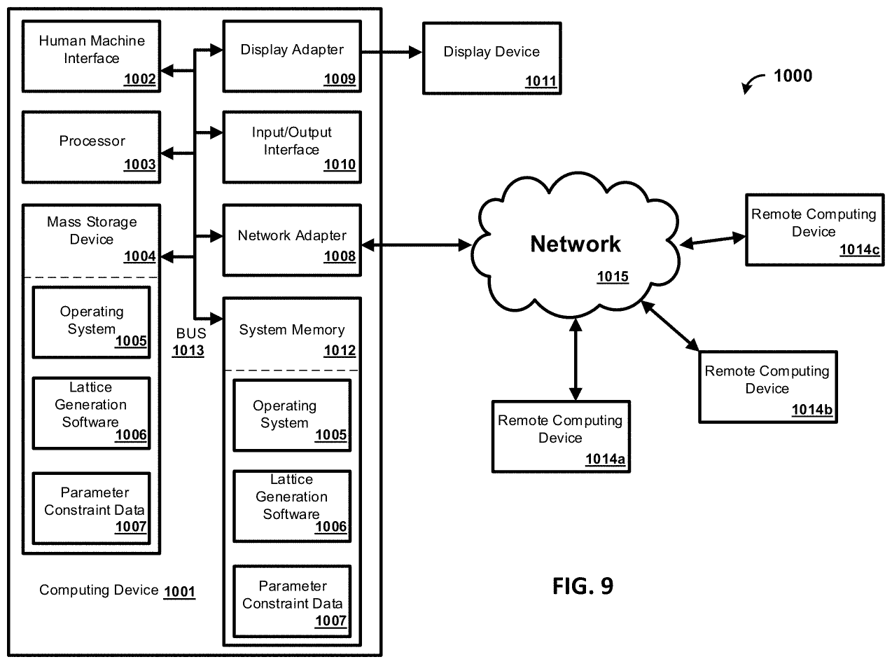

In some aspects, an operator (e.g., a clinician) can manually edit the insole model. For example a computing device 1001 (FIG. 9) can provide an interface that permits the operator to change at least one parameter of the insole model. In some aspects, manually editing the insole model can comprise changing a density of intersections and elongate segments. In some aspects, manually editing the insole model can comprise changing a selected region of the insole model. In some aspects, manually editing the insole model can comprise changing at least one parameter of the insole model.

In some aspects, the lattice can comprise at least one lattice parameter. For example, the at least one lattice parameter can comprise one or more of: an overall material density, an elongate segment length, a density of elongate segments per unit volume, a density of intersections per unit volume, an elongate segment thickness, or an elongate segment cross sectional shape (e.g., round, triangular, rectangular, or hexagonal).

In various aspects, the desired insole can be divided into points or regions. For example, each point or region can have one or more specified material properties (e.g., thickness and/or strain at one or more pressures). The software that is used to generate the lattice can generate a lattice that provides the one or more specified material properties at each point or region.

In some aspects, software can be used to generate the lattice. For example, certain design parameters (e.g., patient specific data) can be provided to a computing device, and the software can generate a lattice that forms the insole model. The design parameters can include one or more of: thickness, strain at one or more pressures, weight of the user, or geometry (e.g., points or regions with pressures associated therewith).

The method can further comprise forming the insole based on the insole model (e.g., by 3D printing the insole).

Disclosed herein, in one aspect, is a method comprising forming, using additive manufacturing and an insole model based on a profile of a foot and a patient-specific pressure distribution, an insole.

In one aspect, an insole can comprise a base formed by additive manufacturing. The shoe sole can further comprise at least one additional layer that is coupled to the base.

### Computing Device

FIG. 9 shows a computing system 1000 including an exemplary configuration of a computing device 1001 for use with the systems and methods disclosed herein. The computing system can generate an insole model. In some aspects, the computing system 1000 can be in communication with a 3D printer.

In exemplary aspects, the computing device 1001 can receive the profile (e.g., 3D geometry) of the patient's foot and plantar pressure measurements (e.g., standing, walking, or both). In some aspects, the computing device 1001 can determine the shape of the insole based on the profile of the patient's foot. In other aspects, the shape of the insole can be based on predetermined parameters as a function of a measurement such as shoe size. In other aspects, the shape of the insole can be input by an operator. The computing device 1001 can generate an insole model. For example, the insole model can comprise the geometry of the lattice 20 (FIG. 1C). In some aspects, the computing device 1001 can provide an interface for modifying the insole model. For example, the interface can permit an operator to modify the outer profile of the insole model and/or the parameters that affect strain in certain regions of the insole.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as parameter constraint data 1007 (i.e., data from signals received by the wireless sub) and/or program modules such as operating system 1005 and lattice generation software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and lattice generation software 1006 may be stored on the mass storage device 1004. One or more of the operating system 1005 and lattice generation software 1006 (or some combination thereof) may comprise program modules and the lattice generation software 1006. The parameter constraint data 1007 may also be stored on the mass storage device 1004. The parameter constraint data 1007 may be stored in any of one or more databases known in the art. The databases may be centralized or distributed across multiple locations within the network 1015.

A user may enter commands and information into the computing device 1001 using an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In some optional aspects, the remote computing devices 1014a,b,c can be in direct communication with each other and the computing device 1001. In various further aspects, it is contemplated that some or all aspects of data processing described herein can be performed via cloud computing on one or more servers or other remote computing devices. Accordingly, at least a portion of the system 1000 can be configured with internet connectivity.

### EXEMPLARY METHODS

### Example 1

Novel fabrication methods disclosed herein achieve a fully customized insole that fits the patient's foot and shoe, as prescribed by an orthotist, and incorporates patient-specific plantar pressure to relieve plantar pressure through targeted offloading regions of insole stiffness reduction. The insoles created using the novel fabrication methods are referred to herein as the novel insole.

Novel fabrication methods disclosed herein can achieve a fully customized insole that fits the patient's foot and shoe, as prescribed by an orthotist, and incorporates patient-specific plantar pressure to relieve plantar pressure through targeted offloading regions of insole stiffness reduction. The insoles created using the novel fabrication methods are referred to herein as the novel insole. A foam crush box impression of the patient's foot generated by an orthotist was scanned using a high-precision 3D visible light scanner (Creaform GoScan50, Creaform, Levis, Canada). The scanned data was processed using a scan post-processing software (VX Elements 3D, Creaform, Levis, Canada) and exported the impression as an STL file for later patient-specific insole design. Patient-specific plantar pressure was collected using an in-shoe plantar pressure sensor (Pedar-X, novel GMBH, Germany) while walking over flat ground in a laboratory setting in a standardized shoe. The plantar pressure data were calculated using a custom MATLAB (The MathWorks Inc., Natick, Mass.) algorithm to define the offloading regions using a threshold value of 200 kPa. In insole model design software (FitFoot360, Fit360 Ltd., Worcestershire, UK), the anatomical landmarks of the foot, including heel and first and fifth metatarsal heads, were manually identified to facilitate proper insole positioning and sizing, and the insole was modeled to match the geometry of the patient's scanned foot. The resulting one-piece insole model was then exported and further divided into normal-pressure and offloading segments based on a previously defined plantar pressure map using CAD software (Fusion360, Autodesk Inc. San Rafael, Calif.). One version of the model was exported incorporating the full thickness of the model produced through FitFoot360, and a separate model with the top 4 mm of thickness removed was also exported. These two segmented models were then delivered to a third-party company (Carbon, Redwood City, Calif.) for application of the lattice structure 3D printing.

After extensive testing of a range of elastomers available for various 3D printing technologies, the Carbon® Elastomeric Polyurethane (EPU) 41 material was identified to exhibit the most suitable mechanical properties in terms of elasticity and durability. Furthermore, the lattice design engine (Carbon, Redwood City, Calif.) enabled creation of a lattice with comparable stiffness to the foam materials used in the SoC insoles by iteratively adjusting the lattice unit size and strut thickness. During the lattice tuning phase, the predicted lattice stiffness was generated by the design engine [26]. Lattice unit size and thickness parameters were tuned until the predicted stiffness resembled the characterized stiffnesses of each of the SoC insole materials. The resulting printed samples were tested for compressive stiffness and the process was repeated until the lattice parameters successfully produced a stiffness closely matching those of the SoC materials. A resulting material with stiffness-matched regions produced according to each individual layer of the SoC insole foam was delivered through the capability of the lattice design engine. Plots of compressive stiffness verification for the final lattice designs compared to the matching SoC foams can be seen in the included supplementary documents, as well as figures detailing the lattice structure. The designs of the full insole models including the latticed structures were then 3D printed on a Carbon L1 3D printer using the EPU41 material. Following the standard Carbon fabrication process for the L1 printing system, once printed, the insoles were spun in a centrifuge to remove excess resin and finished with a heat cure process in an oven. For a hybrid version, a thin base of the insole was printed using this process, and the thin base sole was bonded to a Poron-Plastazote bi-laminate top sheet, nominally 4 mm in thickness, to attain the same overall geometry of the fully-3D printed version, and to target a surface feel preferred by patients and clinicians. This Poron-Plastazote bi-laminate top sheet is the same that is used for the SoC customized insoles (FIG. 1B).

Material Mechanical Property Characterization

Durability and Compressive Stiffness Characterization

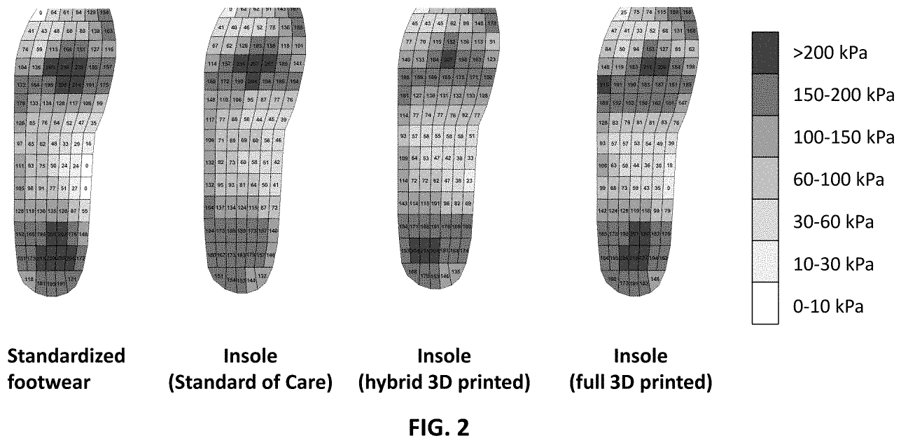

3 cylindrical specimens (nominally 31.75 mm in diameter and approximately 8 mm thick, FIG. 2A) were prepared, two of which were samples of the novel insoles, either consisting entirely of EPU41 in lattice geometry manufactured by Carbon or with the additional bi-laminate foam top layer consisting of a soft Plastazote® layer (Zotefoams, Croydon, UK) and a stiffer PORON® layer beneath (Rogers Corporation, Chandler, Ariz.). The last specimen was a SoC tri-layer foam (Plastazote, PORON, and EVA 35A) manufactured by Amfit Inc. (Vancouver, Wash.). Those specimens underwent a cyclic compressive loading using a material testing machine (Instron, Norwood, Mass., FIG. 2).

Each specimen was loaded cyclically using a sinusoidal loading pattern from zero to peak load to simulate an expected walking task. Specifically, a loading protocol with a total cycle count of 1 million at a rate of 1.1 Hz and a compressive pressure of 300 kPa was designed based on the fact that a standard pattern of use was determined to be 5,400 steps per day, or 985.5K steps per insole in 12 months (FIG. 2) [27-29]. Standard of care insoles are prescribed for a maximum use equivalent to 6 months of total wear. However, all samples were tested for a simulated period of 1 year in order to ensure safety prior to using in a clinical application.

Data were processed to extract stress and strain for each recorded cycle, and the compressive elastic modulus of the sample was determined at each cycle by the following method: the linear region of the stress vs. strain curve was approximated to be contained within the last 25% of the data, then the slope of this linear region was estimated by fitting a first-order polynomial using the Python (Python Software Foundation, Wilmington, Del.) Numpy package's polyfit function, which uses a least-squares polynomial fit method. This slope estimation was calculated for each recorded cycle and the change in this slope value was used as a means of comparing the tendency of each insole construction method sample to become stiffer over the course of the loading protocol. This method of characterizing and comparing the compressive modulus was used because the highest pressures felt by the plantar tissue can be experienced in the last portions of the stress-strain curve. Therefore, this can be the most relevant region in which to assess the pressure-reducing ability of a particular insole material. This method was used in several prior works and standardized testing methods [30,31].

Shear Stiffness Characterization

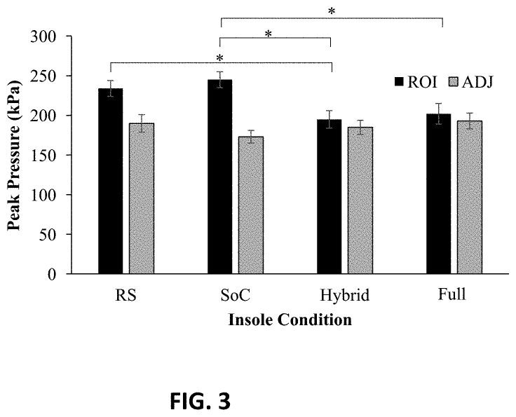

To characterize the novel insole's ability to mitigate shear stress of the plantar tissue, the shear stiffnesses of the SoC insole and variations of the two 3D printed insole construction methods were characterized using a Mach-1 Mechanical Testing Machine (Biomomentum Inc., Laval, QC, FIG. 3A). Cylindrical samples (nominally 31.75 mm in diameter and 8 mm in thickness) were prepared for the following insole constructions: 1) SoC EVA with Poron-Plastazote bi-laminate top sheet; 2) 3D printed nominal stiffness lattice-matched to SoC stiffness profile (either fully-3D printed or of hybrid construction with Poron-Plastazote bi-laminate top sheet); 3) 3D printed sparse lattice matching an offloading region stiffness (either fully-3D printed or with Poron-Plastazote bi-laminate top sheet).

A loading protocol was applied to a previously untested sample of each construction via the Mach-1 Motion Software wherein the sample was loaded in compression to a pressure of 50 kPa, and subsequently loaded cyclically in shear to a strain of 6% for a total of 100 cycles at a rate of 1.1 Hz (FIG. 3B). The duration of this preconditioning stage was predetermined by subjecting a prior selection of samples to a total of 1,000 shear loading cycles and calculating the change in shear stiffness versus cycle count. It was found that after 100 cycles, all samples exhibited a change in shear stiffness of less than 1% per cycle. Therefore, this was set as the threshold for shear preconditioning.

Testing data for the last shear loading cycle of each sample was recorded, including shear load and shear displacement. These data, as well as sample geometry, were used to compare shear stiffnesses (G) between samples. Shear stress was calculated as the ratio of shear load to sample cross-sectional area, and shear strain was calculated as the ratio of change in shear displacement to sample thickness. In order to avoid slight nonlinear stiffness behavior in the initial loading stage, the last 25% of the load cycle was isolated and the slope of the shear strain vs. shear stress data was calculated using the Python Numpy package's built-in Polyfit function. This method was informed by the same process as described in the compressive stiffness characterization method above.

Application Case Study

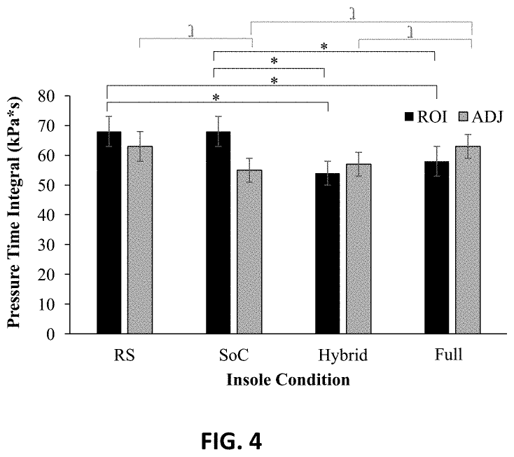

A case study with one participant with diabetes without neuropathy or previous ulceration was carried out (male, type II diabetes for 6-7 years, age 71, body height: 199 cm, body mass: 109.4 kg, BMI: 27.6 kg/m2). Each of the insole manufacturing procedures detailed above were followed to design three different pairs of insoles for the participant: one SoC, one hybrid 3D printed insole with a bi-laminate foam top layer, and one fully-3D printed insole. An orthotist performed the foot evaluation and foam crush box impression. The foam crush box impression was used for the SoC insole manufacturing by one of the hospital's existing third-party vendors. In this case, no offloading regions were defined for the SoC insoles based on clinical evaluation and judgments (FIG. 4). The same impression was also 3D scanned for the creation of the hybrid and fully-3D printed insole designs, and the participant underwent an in-shoe plantar pressure measurement. The plantar pressure map was generated, and an offloading region was defined for this case based on the preset cut-off pressure of 200 kPa (FIG. 4).

After all three pairs of insoles were manufactured, the participant was asked to walk 40 ft in a straight line with one insole type at a time, and the in-shoe plantar pressure measurement was performed again. We collected multiple gait trials until we had 4 successful trials for each insole condition. To avoid any potential effect of fatigue, a rest session and a warm-up session was introduced between each insole condition. Gait speed of each gait trial was recorded using a stopwatch and a variation of less than 10% was used to define a successful trial. Each collected set of plantar pressure data was post-processed to segment it into steps. The steps that occurred during gait initiation and termination phases were removed from the analysis. The peak pressure of each sensor cell during each step was found first and then the peak pressures were calculated across all steps in each gait trial. Maximum peak pressure, defined as the highest peak pressure in the offloading region and adjacent region, defined by one cell apart from the offloading region, was calculated from the data to demonstrate the biomechanical effect of SoC and 3D printed insoles.

Results

Material Mechanical Property Characterization

In the durability and compressive stiffness testing, the hybrid 3D printed insole sample closely matched the durability profile of the SoC insole sample, whereas the fully-3D printed insole sample exhibited a lower increase in stiffness over the course of 1 million cycles. At cycles 1K, 10K, 100K, and 1M, the fully-3D printed sample showed a percent deformation of the original thickness of 3.88%, 4.30%, 5.45%, and 9.49%, respectively. At the same cycle counts, the hybrid sample showed deformation of the original thickness of 9.91%, 20.58%, 24.13%, and 25.20%, respectively. Lastly, for the same cycle counts, the SoC sample showed deformation of the original thickness of 10.74%, 21.08%, 23.59%, and 25.42%, respectively.

In terms of the shear stiffness (G) for each insole sample, the SoC insole sample had the highest shear stiffness of 223 kPa, followed by the fully-3D printed nominal lattice sample (191 kPa), the hybrid nominal lattice sample (160 kPa), the hybrid sparse lattice sample (122 kPa), and finally the fully-3D printed sparse lattice sample (109 kPa), demonstrating that the SoC had the highest stiffness at a given shear displacement over the range of tested shear strain.

Application Case Study

The SoC insole without any offloading region showed an increase in peak plantar pressure compared to the standardized footwear (Mean±Standard deviation: 268.8±7.0 kPa vs. 248.8±9.9 kPa) during walking. The maximum peak plantar pressure values for the hybrid and fully-3D printed insoles in the offloading region were 207.8±9.6 kPa (˜16.5% reduction vs. standardized footwear) and 209.3±2.9 kPa (˜15.9% reduction vs. standardized footwear). In the region adjacent to the offloading region, the SoC reduced the peak pressure during gait to 161.0±4.9 kPa compared to the SoC insole of 186.7±13.8 kPa. The maximum peak plantar pressure values for the hybrid 3D printed, and fully-3D printed insoles were 187.8±10.0 and 213.5±15.8 kPa.

### Discussion

A 3D printed lattice material that matched the SoC compressive stiffness for the bilaminate foam and EVA foam was produced; an insole material with improved durability over the SoC material was developed; the shear stiffness of the insole material over the SoC material was reduced; the customization of insole stiffness based on patient plantar pressure data was allowed; and a repeatable workflow for the manufacturing of patient-specific 3D printed plantar pressure-offloading insoles and for the rigorous characterization of new 3D printed metamaterial samples for future insole development was developed.

A novel method to design and manufacture 3D printed accommodative insoles with patient-specific metamaterials is presented herein. A benchtop and human subjects testing framework is disclosed through which the impact of design parameters on the performance of the device can be assessed in a scientifically rigorous manner. The ability to control nominal lattice geometry and stiffness as well as offloading regional geometry and stiffness was demonstrated. The workflow also facilitated the comparison of printing resins, foam materials, and the combinations of each of these constituent components. A testing protocol is provided for compressive stiffness, compressive durability, and shear stiffness through which the 3D printed lattice parameters can be evaluated and tuned to produce desirable results. The application of a selected elastomeric 3D printing material, EPU41, and a printing process, Carbon DLS™, within the fabrication and material characterization workflow by producing two complete sets of patient-specific insoles (in fully-3D printed and hybrid construction) and comparing the resulting plantar pressure measurements with those of the SoC insole has been demonstrated.

Durability of an insole product is a key performance metric regarding its potential clinical effectiveness [32-34]. The ability of an insole device to mitigate plantar pressure can change as the device stiffens, and therefore the ability of an accommodative insole to retain its soft elasticity is critical. The deformation pattern of the disclosed 3D printed insole shows that the accommodative behavior with the SoC can be fully simulated, such as the hybrid version, and a different profile, such as the fully-3D printed version, can be created. A key difference between the two novel insoles is the presence of the Plastazote top layer, which is specifically intended to compress quickly over areas of high pressure, thereby creating reliefs in areas of peak pressure. The decision to produce a novel insole with Plastazote and one without permitted investigation of the effect of this material on overall durability performance. Furthermore, prior studies in the area of novel 3D printed insole devices have not demonstrated an evaluation of the insoles' durability, either alone or in comparison to SoC insoles [12-15,21]. After subjecting representative samples for the two novel insole variations and SoC insole to a 1 million cycle compressive loading test, it has been demonstrated that the two novel insoles were at least as durable as the SoC for a year's worth of steps for an average patient with diabetes and that the fully-3D printed insole actually demonstrated a lower increase in stiffness, suggesting a higher durability performance. Therefore, the comfort level of the insole from a patient perspective may match or exceed the SoC insole, which may augment patient adherence to a treatment plan that includes regular use of the insole [35-37].

The 3D printed insole fabrication method and material selection showed the potential to reduce shear stress on the plantar tissue of the wearer by reducing the shear stiffness of the fabricated insole. The SoC construction yielded the highest shear stiffness, followed by the standard-stiffness 3D printed sample and softest-stiffness offloading region 3D printed sample, suggesting that the novel insole product has the ability to regionally control shear stiffness and compressive stiffness to accommodate patient-specific requirements. Plantar shear-offloading for patients with diabetes has been investigated for its potential to reduce rates of plantar ulceration.

Several variables can be controlled within the workflow: printing resin, lattice geometry, offloading region geometry, and offloading region stiffness. One sample of each insole construction method was tested as a proof of concept for the novel fabrication workflow and testing protocols. Durability testing was also performed via a benchtop simulation of 1 year of use. The impact of design variables, such as 3D printed lattice variables (cell size, infill percentage, printing resin), offloading region design (geometry, nominal-sparse transition method, offloading stiffness), and upper foam selection through clinical evaluation can be determined. Also, the insole manufacturing workflow presented in this study can be used for other foot conditions, such as rheumatoid arthritis, osteoarthritis, plantar fasciitis, and metatarsalgia, that require custom geometry and a mixture of materials to achieve pain relief or correction.

The case study results indicate that the 3D printed insole, informed by the plantar pressure mapping workflow, successfully offloaded plantar pressure in the desired region as designed with minimal secondary adjustment or fabrication steps. This presents an important improvement in clinical applications by shortening the clinical time for the patient to a single visit. In addition, with the 3D printing technology disclosed herein, multiple insoles can be manufactured simultaneously if a patient has multiple types of footwear. Although manually fabricated insoles have shown clinical efficacy, the strength of 3D printing technology enables further redesign and optimization when newer materials are available for clinical use. The disclosed workflow can be used to assess new 3D printed insole material samples for further development and refinement of this novel 3D printed insole technology.

### REFERENCES

The following references are hereby incorporated by reference herein in their respective entireties.
[1] D. F. Bandyk, The diabetic foot: Pathophysiology, evaluation, and treatment, Semin. Vasc. Surg. 31 (2018) 43-48. https://doi.org/10.1053/j.semvascsurg.2019.02.001.
[2] B. A. Lipsky, É. Senneville, Z. G. Abbas, J. Aragón-Sánchez, M. Diggle, J. M. Embil, S. Kono, L. A. Lavery, M. Malone, S. A. van Asten, V. Urbančič-Rovan, E. J. G. Peters, on behalf of the I. W. G. on the D. Foot (IWGDF), Guidelines on the diagnosis and treatment of foot infection in persons with diabetes (IWGDF 2019 update), Diabetes Metab. Res. Rev. 36 (2020) e3280. https://doi.org/10.1002/dmrr.3280.
[3] M. A. Del Core, J. Ahn, R. B. Lewis, K. M. Raspovic, T. A. J. Lalli, D. K. Wukich, The Evaluation and Treatment of Diabetic Foot Ulcers and Diabetic Foot Infections, Foot Ankle Orthop. 3 (2018) 2473011418788864. https://doi.org/10.1177/2473011418788864.
[4] A. Veves, H. J. Murray, M. J. Young, A. J. M. Boulton, The risk of foot ulceration in diabetic patients with high foot pressure: a prospective study, Diabetologia. 35 (1992) 660-663. https://doi.org/10.1007/BF00400259.
[5] S. A. Bus, D. G. Armstrong, R. W. van Deursen, J. E. A. Lewis, C. F. Caravaggi, P. R. Cavanagh, IWGDF guidance on footwear and offloading interventions to prevent and heal foot ulcers in patients with diabetes, Diabetes Metab. Res. Rev. 32 (2016) 25-36. https://doi.org/10.1002/dmrr.2697.
[6] M. L. Maciejewski, G. E. Reiber, D. G. Smith, C. Wallace, S. Hayes, E. J. Boyko, Effectiveness of Diabetic Therapeutic Footwear in Preventing Reulceration, Diabetes Care. 27 (2004) 1774-1782. https://doi.org/10.2337/diacare.27.7.1774.
[7] U. Hellstrand Tang, R. Zügner, V. Lisovskaja, J. Karlsson, K. Hagberg, R. Tranberg, Comparison of plantar pressure in three types of insole given to patients with diabetes at risk of developing foot ulcers— A two-year, randomized trial, J. Clin. Transl. Endocrinol. 1 (2014) 121-132. https://doi.org/10.1016/j.jcte.2014.06.002.
[8] T. M. Owings, J. L. Woerner, J. D. Frampton, P. R. Cavanagh, G. Botek, Custom Therapeutic Insoles Based on Both Foot Shape and Plantar Pressure Measurement Provide Enhanced Pressure Relief, Diabetes Care. 31 (2008) 839-844. https://doi.org/10.2337/dc07-2288.
[9] P. E. Chatzistergos, A. Gatt, C. Formosa, K. Farrugia, N. Chockalingam, Optimised cushioning in diabetic footwear can significantly enhance their capacity to reduce plantar pressure, Gait Posture. 79 (2020) 244-250. https://doi.org/10.1016/j.gaitpost.2020.05.009.
[10]J. J. van Netten, I. C. N. Sacco, L. A. Lavery, M. Monteiro-Soares, A. Rasmussen, A. Raspovic, S. A. Bus, Treatment of modifiable risk factors for foot ulceration in persons with diabetes: a systematic review, Diabetes Metab. Res. Rev. 36 (2020) e3271. https://doi.org/10.1002/dmrr.3271.
[11]L. K. Johnson, C. Richburg, M. Lew, W. R. Ledoux, P. M. Aubin, E. Rombokas, 3D Printed lattice microstructures to mimic soft biological materials, Bioinspir. Biomim 14 (2018) 016001. https://doi.org/10.1088/1748-3190/aae10a.
[12]S. Telfer, J. Woodburn, A. Collier, P. R. Cavanagh, Virtually optimized insoles for offloading the diabetic foot: A randomized crossover study, J. Biomech. 60 (2017) 157-161. https://doi.org/10.1016/j.jbiomech.2017.06.028.
[13]Z. Ma, J. Lin, X. Xu, Z. Ma, L. Tang, C. Sun, D. Li, C. Liu, Y. Zhong, L. Wang, Design and 3D printing of adjustable modulus porous structures for customized diabetic foot insoles, Int. J. Lightweight Mater. Manuf. 2 (2019) 57-63. https://doi.org/10.1016/j.ijlmm.2018.10.003.
[14]S. Jandova, R. Mendricky, Benefits of 3D Printed and Customized Anatomical Footwear Insoles for Plantar Pressure Distribution, 3D Print. Addit. Manuf. (2021). https://doi.org/10.1089/3dp.2021.0002.
[15]A. Peker, L. Aydin, S. Kucuk, G. Ozkoc, B. Cetinarslan, Z. Canturk, A. Selek, Additive manufacturing and biomechanical validation of a patient-specific diabetic insole, Polym. Adv. Technol. 31 (2020) 988-996. https://doi.org/10.1002/pat.4832.
[16]B. Rankouhi, S. Javadpour, F. Delfanian, T. Letcher, Failure Analysis and Mechanical Characterization of 3D Printed ABS With Respect to Layer Thickness and Orientation, J. Fail. Anal. Prev. 16 (2016). https://doi.org/10.1007/s11668-016-0113-2.
[17]B. Banjanin, G. Vladic, M. Pal, S. Balos, M. Dramicanin, M. Rackov, I. Knezevic, Consistency analysis of mechanical properties of elements produced by 1-DM additive manufacturing technology, Matér. Rio January 23 (2018). https://doi.org/10.1590/51517-707620180004.0584.
[18]R. Zou, Y. Xia, S. Liu, P. Hu, W. Hou, Q. Hu, C. Shan, Isotropic and anisotropic elasticity and yielding of 3D printed material, Compos. Part B Eng. 99 (2016) 506-513. https://doi.org/10.1016/j.compositesb.2016.06.009.
[19]T. Lim, H. Cheng, W. Song, J. Lee, S. Kim, W. Jung, Simulated and Experimental Investigation of Mechanical Properties for Improving Isotropic Fracture Strength of 3D-Printed Capsules, Materials. 14 (2021) 4677. https://doi.org/10.3390/ma14164677.
[20]K. Yildiz, F. Medetalibeyoglu, I. Kaymaz, G. R. Ulusoy, Triad of foot deformities and its conservative treatment: With a 3D customized insole, Proc. Inst. Mech. Eng. [H]. 235 (2021) 780-791. https://doi.org/10.1177/09544119211006528.
[21]R. Teixeira, C. Coelho, J. Oliveira, J. Gomes, V. V. Pinto, M. J. Ferreira, J. M. Nobrega, A. F. da Silva, O. S. Carneiro, Towards Customized Footwear with Improved Comfort, Materials. 14 (2021) 1738. https://doi.org/10.3390/ma14071738.
[22]M. Hossain, R. Navaratne, D. Perić, 3D printed elastomeric polyurethane: Viscoelastic experimental characterizations and constitutive modelling with nonlinear viscosity functions, Int. J. Non-Linear Mech. 126 (2020) 103546. https://doi.org/10.1016/j.ijnonlinmec.2020.103546.
[23]J. Morita, Y. Ando, S. Komatsu, K. Matsumura, T. Okazaki, Y. Asano, M. Nakatani, H. Tanaka, Mechanical Properties and Reliability of Parametrically Designed Architected Materials Using Urethane Elastomers, Polymers. 13 (2021) 842. https://doi.org/10.3390/polym13050842.
[24]Y. Jiang, Q. Wang, Highly-stretchable 3D-architected Mechanical Metamaterials, Sci. Rep. 6 (2016) 34147. https://doi.org/10.1038/srep34147.
[25] DLS 3D Printing Technology, Carbon. (n.d.). https://www.carbon3d.com/carbon-dls-technology/(accessed Feb. 8, 2022).
[26]Carbon Design Engine—A New Way to Design 3D Printed Products, Carbon. (n.d.). https://www.carbon3d.com/products/carbon-design-engine/(accessed Feb. 8, 2022).
[27]D. G. Armstrong, L. A. Lavery, H. R. Kimbriel, B. P. Nixon, A. J. M. Boulton, Activity Patterns of Patients With Diabetic Foot Ulceration: Patients with active ulceration may not adhere to a standard pressure off-loading regimen, Diabetes Care. 26 (2003) 2595-2597. https://doi.org/10.2337/diacare.26.9.2595.
[28]A.-S. Brazeau, S. Hajna, L. Joseph, K. Dasgupta, Correlates of sitting time in adults with type 2 diabetes, BMC Public Health. 15 (2015) 793. https://doi.org/10.1186/s12889-015-2086-6.
[29]K. Dasgupta, L. Joseph, L. Pilote, I. Strachan, R. J. Sigal, C. Chan, Daily steps are low year-round and dip lower in fall/winter: findings from a longitudinal diabetes cohort, Cardiovasc. Diabetol. 9 (2010) 81. https://doi.org/10.1186/1475-2840-9-81.
[30]J. D. Lord, R. Morrell, Measurement Good Practice Guide No. 98 Elastic Modulus Measurement, (n.d.) 100.
[31]L. A. Mihai, A. Goriely, How to characterize a nonlinear elastic material? A review on nonlinear constitutive parameters in isotropic finite elasticity, Proc. Math. Phys. Eng. Sci. 473 (2017) 2017 0607. https://doi.org/10.1098/rspa.2017.0607.
[32]A. C. Faulí, C. L. Andrés, N. P. Rosas, M. J. Fernandez, E. M. Parreño, C. O. Barceló, Physical Evaluation of Insole Materials Used to Treat the Diabetic Foot, J. Am. Podiatr. Med. Assoc. 98 (2008) 229-238. https://doi.org/10.7547/0980229.
[33]J. S. Paton, E. Stenhouse, G. Bruce, R. Jones, A Longitudinal Investigation into the Functional and Physical Durability of Insoles Used for the Preventive Management of Neuropathic Diabetic Feet, J. Am. Podiatr. Med. Assoc. 104 (2014) 50-57. https://doi.org/10.7547/0003-0538-104.1.50.
[34]J. W. Brodsky, F. E. Pollo, D. Cheleuitte, B. S. Baum, Physical Properties, Durability, and Energy-Dissipation Function of Dual-Density Orthotic Materials Used in Insoles for Diabetic Patients, Foot Ankle Int. 28 (2007) 880-889. https://doi.org/10.3113/FAI.2007.0880.
[35]S. A. Bus, R. W. M. van Deursen, R. V. Kanade, M. Wissink, E. A. Manning, J. G. van Baal, K. G. Harding, Plantar pressure relief in the diabetic foot using forefoot offloading shoes, Gait Posture. 29 (2009) 618-622. https://doi.org/10.1016/j.gaitpost.2009.01.003.
[36]E. Chantelau, T. Kushner, M. Spraul, How Effective is Cushioned Therapeutic Footwear in Protecting Diabetic Feet? A Clinical Study, Diabet. Med. 7 (1990) 355-359. https://doi.org/10.1111/j.1464-5491.1990.tb01404.x.
[37]E. M. Macdonald, B. M. Perrin, N. Hyett, M. I. C. Kingsley, Factors influencing behavioural intention to use a smart shoe insole in regionally based adults with diabetes: a mixed methods study, J. Foot Ankle Res. 12 (2019) 29. https://doi.org/10.1186/s13047-019-0340-3.
[38]S. Pai, W. R. Ledoux, The shear mechanical properties of diabetic and non-diabetic plantar soft tissue, J. Biomech. 45 (2012) 364-370. https://doi.org/10.1016/j.jbiomech.2011.10.021.
[39]L. Brady, S. Pai, J. M. Iaquinto, Y.-N. Wang, W. R. Ledoux, The compressive, shear, biochemical, and histological characteristics of diabetic and non-diabetic plantar skin are minimally different, J. Biomech. 129 (2021) 110797. https://doi.org/10.1016/j.jbiomech.2021.110797.
[40]J. S. Wrobel, P. Ammanath, T. Le, C. Luring, J. Wensman, G. S. Grewal, B. Najafi, R. Pop-Busui, A Novel Shear Reduction Insole Effect on the Thermal Response to Walking Stress, Balance, and Gait, J. Diabetes Sci. Technol. 8 (2014) 1151-1156. https://doi.org/10.1177/1932296814546528.

### Example 2

### Introduction

Diabetic foot ulcers contribute to a significant loss of life and place a large financial burden on the healthcare system. Over 30 million Americans, or 9.4% of the US population, live with diabetes (Centers for Disease Control and Prevention, 2017); this is projected to be 33% of the population by 2050 (Boyle et al., 2010). From this cohort, over 108,000 non-traumatic amputations were performed in 2014 alone (Centers for Disease Control and Prevention, 2017). Further, 84% of diabetic limb amputations are preceded by ulceration (Pecoraro et al., 1990). Preventing foot ulceration in this high-risk group can be a very effective strategy to reduce the number of lower limb amputations (Apelqvist and Larsson, 2000; Pecoraro et al., 1990).

Foot ulcer treatment and prevention strategies are formulated on the basis of pressure redistribution and reduction of mechanical loading at the ulcer site (Bus et al., 2016; Lewis and Lipp, 2013). To decrease the risk of ulceration and resulting lower limb amputation, custom insoles are commonly prescribed with appropriate shoes to patients with diabetes who are at risk of developing plantar ulcers. Custom insoles aim to reduce aberrant plantar pressures through a soft accommodating material with a weight-bearing surface conformal to the patient's foot that offloads pressure from at-risk areas. Custom diabetic insoles are one of the non-surgical approaches and have been demonstrated to mechanically offload the healing area (Bus et al., 2013; Lavery et al., 2012; Petre et al., 2013; Ulbrecht et al., 2014; Viswanathan et al., 2004).

The manufacturing process for the current standard of care (SoC) insoles often requires hand craftsmanship, leading to variability in their design and therapeutic performance. SoC insoles are generally constructed from open and closed cell foams shaped to match the patient's foot. The material properties of these insoles are generic, which limits the clinician's ability to optimize the device's pressure reduction capability at the patient-specific level.

3D printing, materials, and software enable the disclosed latticed 3D printed metamaterials whose properties are derived not only from the base material but also from the lattice microstructures within the metamaterial. The concept of 3D printed personalized metamaterials (PMM) can be used for insoles. Insoles manufactured using PMM have not only patient-specific geometry but also patient-specific stiffness and structural behavior.

The purpose of this study is to investigate the effect of the SoC diabetic insoles, two types of 3D printed diabetic insoles with PMM, and no custom insole (research shoe; RS) on walking plantar pressure in healthy adults with typical plantar pressures.

Two types of 3D printed diabetic insoles with PMM were studied. The full 3D printed insole was 3D printed on a Carbon® L1 3D printer using the EPU41 material using a custom lattice structure that mimics the compressive properties of the SoC. The hybrid insole was similarly designed but with the top 4 mm consisting of a Poron-Plastazote bilaminate sheet glued to the surface to attain the same overall geometry of the fully-3D printed version, but with a surface feel of the SC (FIG. 1A). Detailed descriptions and mechanical testing can be reviewed in the concurrently published manuscript, Hudak et al., n.d.

The SoC can change the plantar pressure and pressure-time integral (PTI) compared to the RS. 3D printed insoles can change the plantar pressure, and PTI compared to the SoC.

### Methods

### Participants

Fourteen adults (10 males, 4 females, 3 with diabetes, 62.3±8.8 years, BMI 27.8±4.5 kg/m2) participated in the study. All participants were able to walk unassisted for up to three hours with breaks and did not have (1) Charcot foot, (2) any partial amputation of the foot, (3) existing plantar ulcer(s), (4) inability to walk unassisted for 80 ft (24.3 m), and (5) inadequate cognitive or language function to consent or to participate. All participants signed IRB-approved consent and HIPAA release form prior to data collection.

### Study Procedures

Visit 1

A certified orthotist conducted a clinical foot exam, took foam crush box impressions of the participants' feet, and fitted participants with a standardized shoe (Dr. Comfort® Men's Stallion or Women's Refresh, DJO LLC, Lewisville, Tex.). Participants then completed four 20 m straight-line walking trials at their self-selected walking speed. A stopwatch was used to monitor walking speed and ensure the speed is within 10% variation. In-shoe plantar pressure measurements during walking were recorded (pedar-X, novel GmbH, Munich, Germany). A minimum of 12 steps per foot were recorded at 50 Hz to ensure reproducibility of the measurements (Arts and Bus, 2011).

Insole Fabrication

Three insole types were generated. A SoC insole (FIG. 1A) was fabricated using typical methods described in the concurrently published manuscript Hudak et al., n.d. in which the foam crush box was shipped to a local contracted insole manufacturer, to generate the milled the SoC insoles. The SoC insoles consisted of an EVA base with a Plastazote-Poron foam top layer.

Two pairs of 3D printed insoles, hybrid 3D printed (Hybrid, FIG. 1B), and fully-3D printed (Full, FIG. 1C) insoles were generated following the design and manufacturing workflow described in the concurrently published manuscript Hudak et al., n.d., the same foam crush box impression was 3D scanned and the areas with high plantar pressure defined by the in-shoe plantar pressure mapping with a cut-off peak pressure of 200 kPa in the forefoot identified using a custom MATLAB scripts (The MathWorks Inc., Natick, Mass.). A sparse “soft” lattice metamaterial was placed in those locations, and a regular density lattice metamaterial with matched compressive stiffness was used in the rest of the insole.

Visit 2

After the three insoles (SoC, hybrid, and full) were designed and manufactured, the participant returned to the laboratory and was fitted with each insole in the Dr. Comfort® shoes. A certified orthotist ensured each insole was fit to the participant and the shoe. Minor adjustments were made by the orthotist using traditional grinding methods to improve fit as the orthotist deemed necessary.

As in the first visit, the participant conducted the same four walking trials with each of the four footwear conditions (RS, SoC, Hybrid, and Full) in a randomized order in the Dr. Comfort® shoes with the pedar-X system to measure in-shoe plantar pressure. A trial was considered good if the subject's walking speed is within 10% of their self-selected walking speed. Qualitative questions regarding ease of use and comfort of the devices were asked, and responses were recorded. These questions include “what did you like about this insole?”, “What did you dislike about this insole?”, and “which one of the insoles did you like the best/least and why?”

Further, participants wore the insoles in the laboratory for more than an hour while walking, standing, and taking seated breaks. Physical foot exams were performed between each condition to observe any unwanted high pressure loading areas or early signs of skin tissue irritation which may be caused by this novel device.

### Data Reduction and Process

Plantar pressure data for each walking trial were post-processed into steps; start and stop steps were discarded. The peak pressure of each sensor cell (sensil) was found during each step and then the peak pressures were averaged across all steps. Pressure time integral (PTI) was calculated based on the integral of the peak pressure and time during each step. Maximum peak pressure was defined as the highest peak pressure in the high-pressure region (ROI) for each participant (e.g. circle in FIG. 2) and adjacent (ADJ; within one sensil) region. Left and right steps were included in the data analysis.

A linear mixed effects regression was used to determine if there were differences in peak pressure or PTI (the dependent variable) by footwear type (RS, SoC, Full, Hybrid) within location (ROI vs. ADJ) and by location within footwear type. The model had fixed effects footwear type, location and footwear type by location interactions. Random effects included study participant, side within participant (left, right) and 2-way interaction terms for study participant by shoe and study participant by location to represent random intercepts for study participant and side within participant, and random slope terms for footwear and location.

Omnibus tests for the association between outcome and footwear type at each location and for the location by footwear type interaction were carried out using conditional F-tests. Pairwise differences in outcomes by footwear type (6 comparisons) were estimated for outcomes at each location. p-values was adjusted to account for multiple comparisons. The statistical analyses were carried out using R 4.0.3, and packages lme4, emmeans, tidyverse, multcomp and kableExtra.

### Results

One participant dropped out prior to the visit 2, stating they were too busy, and thirteen participants completed the study. During data processing, data from one participant could not be processed using the standardized scripts; therefore, we only included 12 participants' data (8 males, 4 females, 3 with diabetes, 63.8±9.2 years, BMI 27.9±4.5 kg/m2) in the final analysis.

### Foot Health

In all 24 feet from the 12 participants, no skin blister, or irritation was found in the plantar regions, suggesting no signs of insole malperformance. No participant complained of discomfort associated with the SoC or the two 3D printed versions of insoles during and after testing.

### Maximum Peak Plantar Pressure in ROI

There was a significant difference in maximum peak plantar pressure in the ROI across insole conditions (p<0.001; FIG. 3). Pairwise comparisons indicated that the Hybrid insole significantly reduced peak plantar pressure by 39 kPa (17%) compared to the RS (p<0.003) and 50 kPa (20%) compared to the SoC (p<0.001) and the Full insole significantly reduced peak plantar pressure by 32 kPa (14%) compared to the SoC (p=0.001). No other significant differences were observed.

### Maximum Peak Plantar Pressure in ADJ

No significant differences were observed in the adjacent regions across insole conditions (FIG. 3).

### Mean PTI in ROI

There was a significant difference in PTI in the ROI across insole conditions (p<0.001; FIG. 4). Pairwise comparisons indicated that the Hybrid and Full insole significantly reduced PTI by 14 kPa·s (21%) and 10 kPa·s (15%) compared to the RS (p<0.001 and p=0.015), respectively and by 14 kPa·s (21%) and 10 kPa·s (15%) compared to the SoC (p<0.001 and p=0.005), respectively. No other significant differences were observed.

### Mean PTI in ADJ

There was a significant difference in PTI in the adjacent regions across insole conditions (p<0.001; FIG. 4). Pairwise comparisons indicated that the SoC significantly reduced the PTI by 8 kPa·s (13%) compared to the RS (p=0.007) and by 8 kPa·s (13%) compared to the Full (p=0.023). The Hybrid insole significantly reduced the PTI by 6 kPa·s (10%) compared to the Full (p=0.011). No other significant differences were observed.

### Qualitative Results

When asked to choose their favorite insole between SoC, Hybrid, and Full, 58% (7/12) participants chose the Full and 42% (5/12) chose the Hybrid, no participants chose the SoC as their favorite. Language to described what was liked about the Hybrid and Full were similar, including the 3D printed insoles were “more comfortable”, “had better support”, “were more cushioned”, “airy and light”, “felt cooler”, and “felt like a memory foam mattress”, or “like walking on a cloud”. There were very few dislikes about all the insole types, including the SoC.

### Discussion

The purpose of this example was to investigate the effect of the SoC diabetic insoles, two types of 3D printed diabetic insoles with PMM, and no custom insole (research shoe; RS) on walking plantar pressure in healthy adults with typical plantar pressures. The pressure results supported the hypothesis that the novel 3D printed insoles could change plantar pressure measurements in the defined high plantar pressure as well as the adjacent regions compared to the SoC. Foot examination and user survey confirmed that the novel 3D printed insole devices did not have any detrimental effects for a short period of usage and would be acceptable for future experimental studies.

Biomechanically, the two novel 3D printed insoles are more effective in reducing maximum peak pressure and PTI in areas of high pressure compared to the SoC (FIG. 3). This is not surprising as the 3D printed insoles had regions of sparse lattice in the ROI. However, this is encouraging as even those without increased plantar pressure can show a reduction in plantar pressure with the sparse lattice. Theoretically, the extra load in the high-pressure region could be absorbed by the softer metamaterials in the novel 3D printed insole or could be load-sharing to the adjacent regions. The findings indicate the former, as the peak plantar pressure reduction in the offloading region, did not significantly increase pressure in the surrounding tissues. This is demonstrated by the not significant change in maximum peak pressure in the adjacent regions (FIG. 3). This suggests that the novel 3D printed insole could address the high pressure in defined regions and may not increase the risk in the surrounding tissues.

The PTI of the Hybrid and Full insoles in the ROI showed a similar trend to the peak pressure; however, in the ADJ the PTI of the two 3D printed insoles slightly increased. While the role of PTI in addition to peak pressure has been debated because of the inconsistent findings in its association with plantar ulceration outcome (Bus et al., 2013; Melai et al., 2011; Sattari and Ashraf, 2011), the consideration of time with pressure did provide insights into the future insole designs. For example, the transition zone between the desired offloading and adjacent regions could be further optimized to mitigate the increased PTI in the ADJ. Although the increased PTI in both 3D printed insoles was not statistically significant compared to the offloading region based on the initial finding, in future cases, if an increased PTI in the ADJ is found, the insole can be resigned accordingly using personalized metamaterials.

Prior studies have shown that the custom insoles reduce peak plantar pressure (Arts et al., 2015; Bus et al., 2011; Martinez-Santos et al., 2019; Owings et al., 2008; Paton et al., 2012) or PTI (Mueller et al., 2006; Owings et al., 2008) in the diabetic foot with neuropathy or ulceration. Specifically, Owings et al. (Owings et al., 2008) reported that custom shape and pressure-based insoles showed an offloading effect of 32 and 21% peak pressure reduction and 40 and 34% PTI reduction compared to shape only based insoles in patients with diabetes and neuropathy. In the healthy, typical plantar pressure, population, the SoC appears to provide minimal changes compared to the RS (FIGS. 3-4), inconsistent with previous work conducted on the target population (Arts et al., 2015; Bus et al., 2011; Martinez-Santos et al., 2019; Owings et al., 2008; Paton et al., 2012).

This inconstancy could be due to a variety of factors including 1) in (Owings et al., 2008), barefoot plantar pressure measurements used to design of loading in Owings compared to the in-shoe measurements. 2) this study had healthy subjects with typical plantar pressures and there may be a floor effect of the SoC, it may not be able to (and does not need to) accommodate for normal pressures. An existing increased plantar pressure can allow more room to be changed technically. 3) Reductions in pressure-based insole in (Owings et al., 2008) were compared to SoC not to a no custom insole condition. No comparisons between no insole and insole were conducted. 4) Several studies examined the effects of the standard of care insole over multiple assessments giving the insole time to accommodate to the user. In this study we only had a wear period of up to 20 minutes. The smaller effect in the SoC may be due to the short wear period as the SoC often requires accommodative period to reach its maximum effect. Future work is being conducted to review if this limited effect of the SoC exists in those with diabetes and increased plantar pressure in addition to testing the 3D printed insoles in the target population and the effects for the insoles on foot health and plantar pressure over longer term use.

The advance in 3D printed technology has shown its potential to improve current care. With personalized printing, specific aspects of the insole design including shape and stiffness can be modified. Similar design features have been demonstrated to reduce forefoot ulcer risk in patients with diabetes and neuropathy (Ahmed et al., 2020). For example, Bus et al. (Bus et al., 2013) utilized in-shoe plantar pressure measurement to define offloading region with multiple manual modifications until peak pressure at defined regions offloaded to the desired magnitude. Through the 3D printed design process, multiple pairs of insoles with single or combined features could be designed and manufactured at the same time. During the clinical visit, clinicians can use the biomechanical plantar pressure measurements as a reference to inform their clinical decision, advanced insole modifications, and care for diabetic foot to reduce the risk of ulceration and reulceration.

This example work validates the ability to manufacture the 3DP-PB insoles and demonstrates their ability to reduce plantar pressure more than the standard of care while not significantly increasing pressure in the adjacent regions. Additionally, it has been demonstrated that the ability to modify the 3D printed design to offload certain parts of the foot using plantar pressure data and a patient-specific PMM in the 3D printed insole design.

### Example 3

Exemplary novel, 3D printed custom insoles have been developed and tested. These tests include: 1) a repeated measures controlled trial of functionally optimized insoles for people with rheumatoid arthritis, measuring both patient reported outcomes and mechanics; 2) a study of modular lateral wedge insoles for individuals with medial knee osteoarthritis; and 3) a randomized crossover study of virtually optimized insoles for offloading the at-risk diabetic foot.

Most relevant to the present example is the trial of virtually optimized insoles for the at-risk diabetic foot. This involved the development of an integrated computational modeling process that used patient-specific data and produced a personalized insole design intended to reduce forefoot loading. This work was initiated by the development and evaluation of anatomically simplified FE models of the forefoot. The need for this type of modeling approach was driven by the extensive time required to produce and run personalized FE models of the foot, a limitation that has made this technology clinically inaccessible at the individual patient level. It was found that models using simplified bone geometry based on linear measurements of the patient's anatomy from ultrasound imaging combined with external soft tissue geometry from 3D surface scans were able to adequately simulate forefoot plantar loading mechanics. To generate insole designs, an iterative process based on subtractive and additive geometry changes below and behind the metatarsal heads respectively was performed until the design was predicted to reduce plantar loading at forefoot regions to <200 kPa. The software optimization process typically took around 40 hours of computation time.

With the modeling workflow established, 20 at-risk patients with diabetic neuropathy and elevated plantar pressures (screened for via barefoot pressure assessment) were enrolled. Shape (foam box), loading (in-shoe pressure), anatomy (ultrasound), and plantar tissue material properties (ultrasound) were obtained from each patient. Three insole designs were produced for each participant. The personalized design derived via the modeling workflow was produced using two manufacturing methods: direct milling from a shore A hardness 35 ethylene-vinyl acetate (EVA) material; and 3D printing using hard plastic polylactide (PLA). A clinical prescription for each patient was sent along with the foam box impressions to the study clinic's commercial supplier of diabetic offloading insoles. These insoles were considered the patients' current standard of care and served as the comparator for the study. The offloading performance of the insoles was assessed using in-shoe pressure measurements, and the primary outcome variable was pressure reduction at forefoot regions where elevated loading had been measured during the barefoot pressure assessment.

The results showed that both the milled and 3D printed virtually optimized insoles significantly reduced plantar loading at the target areas. This study demonstrated 1) optimizing insole design via a personalized FE modeling workflow was achievable, and 2) the feasibility of using 3D printing technologies to produce these types of insoles. However, several open questions remain. The therapeutic effect of the devices was achieved only through the modification of shape, without any localized changes to the bulk material. The novelty of the methods proposed in the current study is the ability to change the bulk material properties of the insole according to patient-specific measurements. It is contemplated that insoles with patient-specific shape can reduce plantar pressure; it is further contemplated that insoles with patient-specific shape and patient-specific stiffness (bulk material properties) yield even better results. Reduced plantar pressure via PMM is an important tool for orthotist to have in their toolkit as they design and prescribe insoles for people who have diabetes and are at risk of ulceration.

As mentioned, metamaterials have lattice patterns or other microstructures integrated into the material to control its stiffness or behavior on a macroscale. Metamaterials are extremely advantageous from a 3D printing perspective because they greatly broaden the range of bulk material properties that can be produced. Traditionally, 3D printers made objects using hard plastics, such as those made from polylactide (PLA) or acrylonitrile butadiene styrene (ABS). Only recently have new softer materials become available, sometimes in conjunction with new 3D printing machines and technologies. Today the materials available for plastic 3D printing range from very stiff (3 GPa) which is approaching the stiffness of bone (˜14-20 GPa) down three orders of magnitude to 2.5 MPa (like the stiffness of an eraser). However, this is still an order of magnitude stiffer than the top layer of plastazote foam on a traditional SC offloading insole. Only by designing and leveraging latticed metamaterials have we been able to 3D print materials that have stress-strain properties that match SC insoles.

Methods to design and print different lattice patterns to manufacture metamaterials with specific stress-strain curves have been developed, including nonlinear stress-strain relationships. This was achieved by fabricating lattice patterns into the material (FIG. 5). These microstructures include voids in the material, like a honeycomb, supported by solid walls or pillars. The stiffness of the material is decreased because compressive forces cause the pillars to buckle, rather than compressing the microstructures of the elastic material. As the pillars buckle, the structures also compress in a nonlinear way. This allows for the generation of nonlinear elastic metamaterials from a base material with linear elasticity by varying the geometry of the pillars and voids. Variations in the lattice geometry can be used to match a wide variety of elastic moduli and nonlinear stress-strain profiles. These variations affect the Young's modulus of the resultant simulated tissue.

One of the advantages of 3D printed metamaterials is the ability to not only control the bulk metamaterial stiffness but also to have smooth changes in that stiffness within the 3D printed part. For example, the 3D printed insole can have a stiff rigid heel cup through the application of a dense thick lattice, while at the same time having a soft region under the first metatarsal head via a thin sparse lattice. Because the unit cell size of the lattice is only a few millimeters in size, the macro material properties of the 3D printed insole can change smoothly in a gradual way by making small incremental changes to each lattice cell. In this fashion, the metamaterial can truly be personalized to the individual patient; where each of the hundreds of lattice cells are uniquely designed for that patient; much like a fingerprint, the 3D printed insoles with PMM can be unique to each patient's footprint.

### Development and Refinement of the 3D Printed Diabetic Insole

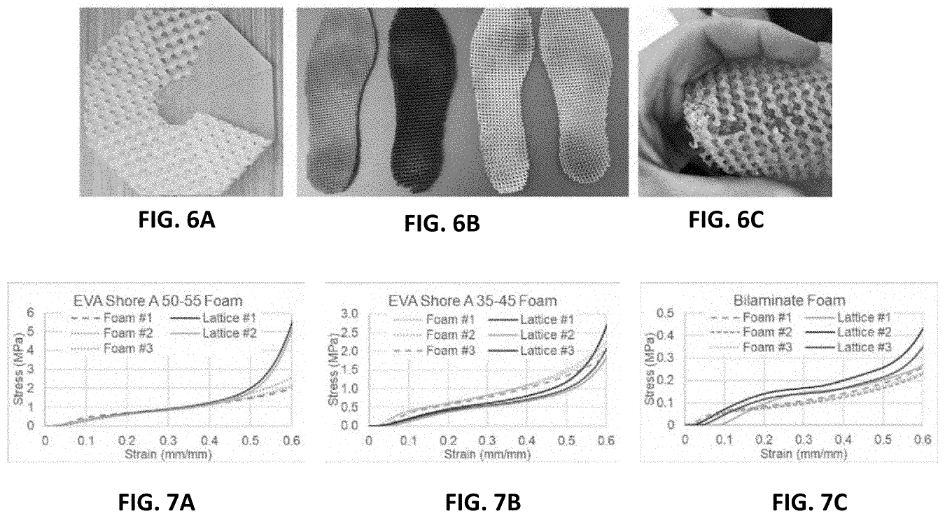

Extensive testing and evaluation of different 3D printing technologies, materials, lattice designs, and 3D printed prototype insoles to find viable solutions that meet the durability, quality, stiffness, and overall insole design requirements have been performed. Pilot work determined materials and lattice designs that were soft enough to replicate foam insoles while also having the durability to withstand one year of typical use. Seventeen commercially available materials and 3D printing technologies that had marketed material properties close to the desired elastomeric properties were investigated. Nine materials were used in 3D printed sample puck-shaped objects with different lattice cell sizes and percent infills (FIG. 6A). For each of the nine different materials, six different pucks were printed, with each puck having a unique unit cell size and six different percent infill combinations. This allowed evaluation of 54 different material lattice combinations (9 materials×6 unit cell sizes) in terms of their metamaterial stiffness and other qualitative properties such as appearance, surface finish, tactile feel, smell, etc. From these 216 different material and lattice combinations five different 3D printed insole prototypes were selected. Rehabilitation medicine clinicians and orthotists provided qualitative feedback on the different materials. These prototypes were also worn by a beta tester for several months during which we monitored for mechanical failures and insole breakdown (FIG. 6C). After more than one million combined beta testing steps, it was concluded that insoles manufactured from elastomeric polyurethane (EPU) 41 material using the Carbon L1 machine (Carbon Inc., Redwood City, Calif.) were the best performing. Testing has shown that 3D printed insoles can last for at least a year of typical use (see also durability testing results in the introduction). Current SC insoles are replaced every four months due to reductions in their offloading efficacy; the 3D printed insoles have the potential to reduce this requirement significantly.

### Lattice Design for the 3D Printed Pressure Based Insole

To design lattice metamaterials that replicated the SC foam insole, the stress strain properties of the three different bodies used to create the SC insoles were characterized. Uniaxial mechanical testing of the EVA shore A 50-55 foam, the EVA shore A 35-45 foam, and the Poron-Plastazote bilaminate sheet was carried out (FIG. 7). Next, a software simulation tool developed by Carbon Inc. (Redwood City, Calif.) was used to simulate the stress strain properties of metamaterials with different lattice patterns.

The software package adjusted the unit cell size and strut thickness of the lattice until the metamaterial stress strain property matched the foam stiffness in simulation. Physical samples of these different lattice designs were then 3D printed on a Carbon L1 3D printer (Redwood City, Calif.) using EPU41 material and compared to their foam counterparts (FIG. 7). Initial results indicated that for strains of up to 50%, which is the maximum expeted strain, the lattice metamaterials replicate the stiffness of the foam samples (FIG. 7). The lattice pattern [that matched the EVA Share A foam was then applied to base of the insole.]

The generic base lattice pattern was then modified according to the patient specific plantar pressure to create a patient specific 3DP-PB insole. The plantar pressure collected in step one described above was used to identify regions of high pressure on the subject's feet. [In this pilot data, high pressure regions were identified using a cut-off threshold of 200 kPa]. A specialized soft sparse lattice pattern, designed to match the stiffness of a typical metatarsal head relief, was then used to fill the region under the third and fourth metatarsal heads. A scaling factor can also be applied to the entire insole lattice pattern based on the subject's weight in order to adjust the global stiffness of the insole to the body weight of the subject. For this subject a scaling factor of 1.0 (no body weight adjustment) was used.

### 3D Printing the Custom Insoles

The 3DP-PB insoles were then 3D printed on a Carbon L1 3D printer (Redwood City, Calif.) using EPU41 material. Following the standard Carbon manufacturing process, once printed the insoles were spun in a centrifuge to remove excess resin and finished with a thermal cure. [The 3D printed insole base was then glued to a Poron-Plastazote bilaminate top sheet to attain the same surface finish preferred by patients and clinicians (FIG. 10)].

### Preliminary Results: 3D Printed Insoles Plantar Pressure

A pilot study with 12 subjects (4 females, 3 subjects with diabetes, 62.5±9 yrs, BMI 28.8±4.6 kg/m2) was conducted. The purpose of the pilot study was to 1) confirm the safety of the 3D printed insole within a healthy population who are not at risk of ulceration, 2) refine and optimize the data collection and 3D printed insole manufacturing processes, and 3) determine the pressure reducing capability of the 3D printed insoles in a healthy population. An ulceration event is a serious health risk for patients with diabetes who are at risk of lower limb amputation. Before allowing the at-risk patient population to wear 3D printed devices in the laboratory for several hours, we wanted to confirm that in a low-risk population they do not cause unwanted high-pressure areas, pain, or irritation on the plantar surface of the foot. Following the design and manufacturing workflow described in section 3.4 above, a certified orthotist took foot impressions of each subject's feet. The crush box was 3D scanned and then shipped to Altheas (Seattle, Wash.), a local VAPSHCS contracted insole manufacturer, to generate the milled SC insoles.

[In-shoe] plantar pressure measurements were taken. Regions of high forefoot plantar pressure were identified and a spare soft lattice was placed in those locations, and the 3DP-PB insole was manufactured. After the two insole types (SC and 3DP-PB) were designed and manufactured, the subject again walked 40 ft in a straight line, within 10% of their self-selected walking speed. The subject wore [a standardized shoe (Dr. Comfort® Men's Stallion or Women's Refresh, DJO LLC, Lewisville, Tex.)] with new insoles and pressure sensing insoles in both shoes. This test was repeated 4 times for the SC and 3DP-PB insole. [Further, participants wore the insoles in the laboratory for more than an hour while walking, standing, and taking seated breaks. Physical foot exams and measured plantar pressure were performed during these procedures to observe any unwanted high pressure loading areas or early signs of skin tissue irritation. In all 24 feet, we found no signs of insole malperformance, nor did any participant complain of discomfort associated with the 3D printed insole. Plantar pressure data were post-processed into steps; start and stop steps were discarded. The peak pressure of each sensil was found during each step and then the peak pressures were averaged across all steps. Pressure time integral was calculated based on the integral of the peak pressure and time during each step. Maximum peak pressure was defined as the highest peak pressure in the high-pressure region (e.g. red circle FIG. 11) and adjacent (within one sensil) region. Left and right steps were included in the data analysis. A repeated measured analysis of variance (ANOVA) (p<0.05) and Tukey-Kramer post-hoc analysis were performed to identify significant differences. Maximum peak pressure and pressure time integral in the high pressure and adjacent regions were analyzed separately. Participants were also asked to provide their qualitative feedback including which device they preferred.

The [maximum] peak plantar pressures of the SC and 3DP-PB insoles [in the high-pressure regions] were analyzed (FIG. 2). [Compared to the standardized shoe condition, the SC insoles showed no significant change in peak plantar pressure (231.4±40.3 kPa vs. 239.8±48.3 kPa, p=0.47], while the 3DP-PB insoles reduced the pressure to [193.1±47.3 kPa (−17% on average, p=0.0002). The pressure time integral showed a similar trend as the maximum peak pressure but did not reach the level of significance (p=0.19). The mean pressure time integral for the SC insoles was 78.5±34.1 kPa·s, 11% higher than the standardized shoe (70.2±16.0 kPa·s) while the mean value for the 3DP-PB insoles was 61.7±19.1 kPa·s (12% reduction on overage), however these results were not statistically significant (p=0.19). In the adjacent regions, the SC and 3DP-PB insoles were not significantly different compared to the standardized shoe. The mean maximum peak plantar pressure was 185.0±41.5, 169.6±31.5, 179.6±30.7 kPa for the standardized shoe, SC insole, and 3D-PB insole, respectively (p=0.66). The mean pressure time integral was 65.5±19.3, 63.8±29.2, 66.8±24.2 kPa·s for the standardized shoe, SC insole, and 3D-PB insole, respectively (p=0.53). All Participants reported preferring the 3DP-PB insoles over the SC. They reported the 3DP-PB insoles were “more comfortable”, “had better support”, “were more cushioned”, and “felt like a memory foam mattress”, or “like walking on a cloud”.] These results demonstrate that softening the 3D printed lattice under the high-pressure regions effectively offloaded the desired area of the foot, while pressure was redistributed into adjacent areas as expected.

### Computational Foot Modeling

Musculoskeletal and FE computational foot modeling have been developed. This model represents 26 bones of the foot and can be used to perform inverse dynamic simulations of foot function in combination with other body components. To construct the model, CT scans were used to create 26 individual geometrical segments. Kinematic links between the bones were simulated using a combination of revolute, universal, and spherical joints. The model is driven using motion capture marker and ground reaction force data. Both cadaveric-based and in vivo FE models have been generated. For the latter study, two subject-specific in vivo FE foot models (normal and diabetic) were created to explore the plantar pressure and internal soft tissue stresses during the stance phase of gait. The model included subject-specific bone, skin, muscle, and fat anatomy obtained from CT and MRI data. Subject-specific hyperelastic material properties, derived from inverse FE analysis of MRI data, were assigned for each soft tissue. Ligaments and tendons were modeled using nonlinear spring and seatbelt elements, respectively. Models underwent rigorous validation against both in vivo experimental data and the literature. The model results suggested the location and time of peak forefoot internal stresses frequently, but not always, coincided with peak plantar pressure. Parametric analysis suggested stiffer plantar fat increased plantar pressure and internal stress. As expected, the diabetic subject had higher stresses beneath the heel (and most other locations), due to stiffer soft tissue properties. In summary, the research team also has substantial and varied experience with developing complex full foot musculoskeletal and FE models.

### Research Design and Methods

A repeated measure study compares biomechanical outcomes (peak plantar pressure and pressure time integral) of two different 3D printed insoles (3DP-PB and 3DP-FE) versus the SC insole with a group of 25 participants who have diabetes, peripheral neuropathy, and elevated barefoot plantar pressure. The 3DP-PB insole has patient-specific shape and PMM designed from the subject's [in shoe] plantar pressure data. The 3DP-FE insoles uses FE simulation to optimize the insoles shape and metamaterial properties to reduce plantar pressures. These three insoles are worn by participants and in-shoe plantar pressure measurements are obtained during steady-state walking. Peak pressure and pressure time integral values are calculated in three regions of the forefoot. Linear mixed effects regression are used to test the following hypotheses:

Tested Hypothesis 1: It is contemplated that the methods disclosed below can be used to confirm that 3DP-PB insoles with PMM designed from a patient's plantar pressure can have a greater reduction in peak plantar pressure and pressure time integral than SC insoles.

Tested Hypothesis 2: It is contemplated that that the methods disclosed below can be used to confirm that 3DP-FE insoles with FE optimization of the insole's surface geometry and PMM lattice properties can have the greatest reduction in peak plantar pressure and pressure time integral as compared to the two other insoles.

### Methods: 3D Printed Standard of Care and Pressure Base Insole Manufacturing

The SC and 3DP-PB insoles were manufactured. The steps are briefly reviewed here.

### 3D Printed Pressure Based Insoles (3DP-PB)

The 3DP-PB insoles were designed from the subject's [in shoe] plantar pressure and foam box impression. The digital 3D scan of the foam crush box impressions can be used to design the plantar surface of the 3DP-PB insoles. Regions of relief (soft sparse lattice) can be placed under areas of high pressure as determined by the subject's [in shoe] walking plantar pressure measurement. The lattice were globally scaled according to the subject's bodyweight using equation 1.

Scale Factor=_{0.8}coerce_{1.2}[1+0.2×(BW−87.8)/(2*16.1)] Eq. 1

Scale Factor is the scaling factor to be applied to the lattice strut thickness, coerce is a function that limits the output to a minimum of 0.8 and a maximum of 1.2, and BW is the subject's body weight. This scaling can allow the overall stiffness of the insole to range from 80% softer to a maximum of 120% stiffer than the typical SC insole based on the subject's weight as compared to the average weight of individuals who are prescribed accommodative insoles (mean 87.8 kg and standard deviation 16.1 kg). Subjects who weigh two standard deviations less than the mean can get insoles 80% as stiff, while subjects who weight two standard deviations more than the mean can get insoles 120% stiff, and proportionally ratioed in between. Once designed, 3DP-PB insoles are printed on a Carbon L1 printer using EPU 41 material. [The 3D printed insole sample is designed to replicate the normal stiffness profile of the SC foam but constructed of a base layer of 3D printed lattice (EPU 41, Carbon®, Redwood City, Calif.) with a bi-layer foam top (Plastazote, Poron).]

### Methods: Finite Element Design of 3D Printed Diabetic Insole

The personalized finite element (FE) model used to perform the virtual simulations is described below briefly.

Model geometry: For each participant, a patient-specific model of the forefoot is generated from the scanned foam impression box and ultrasound measurements of the metatarsal head position and size. The foam impression is trimmed to leave the plantar forefoot tissue, with simplified representations of the metatarsals generated using a CAD program (Rhino v6; Robert McNeel and Associates, WA). Components representing the floor, shoe, and 3DP-FE insole are generated, with geometry and material properties that have been described previously. Anatomical measurements from the ultrasound scans can be used to generate and position simplified rigid geometric representations of the metatarsals that are embedded within the tissue block. Surface models are remeshed as appropriate to ensure relatively even, uniform 2D elements suitable for volumetric meshing. Inter-metatarsal ligaments that restrain the medio-lateral splay of the bones are modelled as tension-only truss elements.

Model meshing: Volumetric meshes are created for the soft tissue component using 15-node tetrahedral elements. Tetrahedral elements have the advantage of allowing fast and simple meshing of complex, organic shapes, and using the 15-node elements has been shown to avoid problems with locking and produce similar results to hexahedral elements with acceptable trade-offs in run time. Hexahedral elements are used to mesh the shoe and insole components. Mesh sensitivity analyses are performed for all components to ensure appropriate element size. Floor and metatarsal components are modeled as rigid bodies.

Boundary conditions and contact interactions: The floor component is rigidly constrained in all directions. The lower surface of the shoe is tied to the floor, and the upper surface is tied to the bottom of the insole. Surface-to-surface contact interactions (coefficient of friction=0.5) between the soft tissue and top surface of the insole can be applied.

Material properties and loading: Load and displacement data from the instrumented shoe can be used to drive an inverse simulation of the FE model (without an insole component) to determine the material properties of the plantar soft tissue using a second order Ogden hyperelastic model. Material constants are initially those from the literature and are adjusted until predicted contact pressures under the second metatarsal head are within 5% of those measured experimentally. Vertical loads are applied to the other metatarsal heads and modified until they match the measured pressures under each head at the instance of peak forefoot loadin. The shoe component are modeled as a hyperfoam material, and the insole modeled as an Ogden or Neo Hooken depending on the specific bulk lattice properties.

Simulations: All FE simulations are set up and performed using FEBio Studio, an NIH-funded, open source FE solver with supporting processing software that was developed specifically for biomechanical applications. The static solver can be used for all simulations.

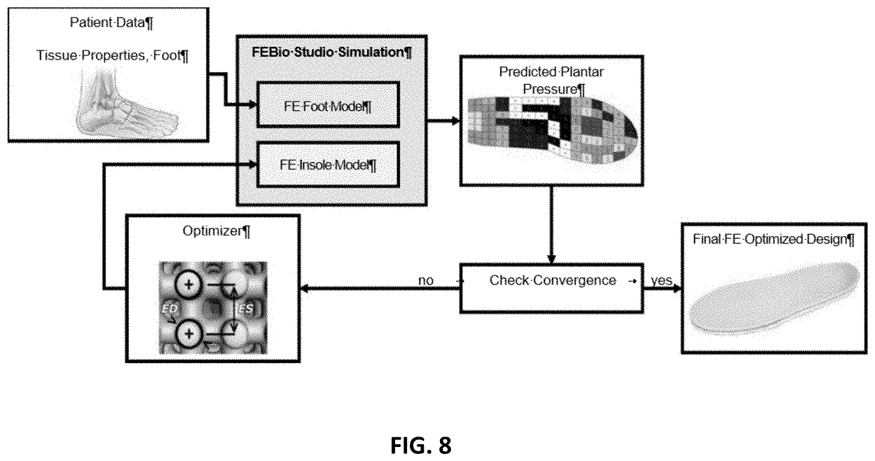

Model output variables: The computational model can yield predictions of plantar pressures below the plantar soft tissue. These can be localized to peak loads under each metatarsal head. An iterative series of simulations can be run for each foot, where the localized material properties of the insole design undergo a standardized modification procedure by reducing the stiffness of the material under each metatarsal head until the limits of reduction in regional peak plantar pressures is reached (FIG. 8). If reducing stiffness underneath the metatarsal heads is predicted to be insufficient to reduce the plantar pressures below the target threshold, a metatarsal bar-type feature can be introduced to the insole geometry. The height of this can be increased after each simulation until the pressures are satisfactorily reduced. Once designed, 3DP-FE insoles can be printed on a Carbon L1 printer using EPU 41 material (Carbon Inc., Redwood, Calif.).

### Methods: Insole Fitting and Plantar Pressure Data Collection

A certified orthotist can fit each patient to an extra depth diabetic shoe, and ensure each insole is fit to the participant and their assigned shoe. Minor adjustments can be made by the orthotist using traditional grinding methods to improve fit as the orthotist deems necessary. Each participant can wear a pair of insoles in a series of walking tests at their previously measured self-selected walking speed. These tests can be repeated until they have worn all versions of the insoles in a randomized order. Subjects can be blinded to the insoles they are wearing.

For each walking test, the subject can be fitted with novel pedar plantar pressure sensors (Munich, Germany), calibrated prior to each data collection following the manufacturer's recommendations. Then, the subject can be asked to walk 80 ft in a straight line (estimating at least 12 steps at a steady speed per foot over this distance). A test is considered good if the subject's walking speed is within 10% of their self-selected walking speed. Walking tests can be repeated until three good trials are collected for each insole type. Subjects can be allowed to rest between walking trials and this time can be used to ask [qualitative questions regarding ease of use and comfort of the devices. These questions include “what did you like/dislike about this insole?”, “which one of the insoles did you like the best/least and why?”, “how was putting the shoe on/taking the shoe off?”, “did you feel any differences in your walking? Please describe.” The certified orthotist can evaluate the plantar surface of the foot between each insole condition to ensure safety.

### Exemplary Aspects

In view of the described devices, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.
Aspect 1: A method comprising:
   generating, based on a profile of a foot and a patient-specific pressure distribution, an insole model.
Aspect 2: The method of aspect 1, wherein the profile of the foot comprises a scan of a crush box impression.
Aspect 3: The method of aspect 2, further comprising scanning the crush box impression.
Aspect 4: The method of any one of the preceding aspects, further comprising forming an insole in accordance with the insole model.
Aspect 5: The method of any one of the preceding aspects, wherein the profile of the foot is a 3-dimensional profile.
Aspect 6: The method of any one of the preceding aspects, further comprising acquiring the patient-specific pressure distribution.
Aspect 7: The method of aspect 6, wherein the patient-specific pressure distribution comprises a reading from a pressure sensing pad.
Aspect 8: The method of any one of the preceding aspects, wherein generating the insole model comprises generating a lattice.
Aspect 9: The method of aspect 8, wherein the lattice comprises a plurality of elongate segments and a plurality of intersections between elongate segments.
Aspect 10: The method of aspect 8, wherein the lattice comprises at least one repeating pattern.
Aspect 11: The method of aspect 10, wherein the at least one repeating pattern comprises hexagonal structures.
Aspect 12: The method of aspect 10, wherein the at least one repeating pattern comprises triangular structures.
Aspect 13: The method of any one of aspects 8-12, wherein the lattice comprises at least one irregular shape.
Aspect 14: The method of any one of the preceding aspects, further comprising manually editing the insole model.
Aspect 15: The method of aspect 14, wherein the lattice comprises a plurality of elongate segments and a plurality of intersections between elongate segments, wherein manually editing the insole model comprises changing a density of intersections and elongate segments.
Aspect 16: The method of aspect 14 or aspect 15, wherein manually editing the insole model comprises changing a selected region of the insole model.
Aspect 17: The method of aspects 8-16, wherein the lattice comprises at least one lattice parameter.
Aspect 18: The method of aspect 17, wherein the lattice comprises a plurality of elongate segments and a plurality of intersections between elongate segments, wherein the at least one lattice parameter comprises one or more of: an overall material density, an elongate segment length, a density of elongate segments per unit volume, a density of intersections per unit volume, or an elongate segment thickness.
Aspect 19: The method of any one of the preceding aspects, further comprising providing an interface to manually edit the insole model.
Aspect 20: The method of any one of the preceding aspects, further comprising forming the insole based on the insole model.
Aspect 21: A method comprising:
   forming, using additive manufacturing and an insole model based on a profile of a foot and a patient-specific pressure distribution, an insole.
Aspect 22: An insole comprising:
   a base formed by additive manufacturing.
Aspect 23: The insole of aspect 22, further comprising: at least one additional layer that is coupled to the base.
Aspect 24: An insole as disclosed herein.
Aspect 25: A shoe comprising an insert sole as disclosed herein.
Aspect 26: A shoe comprising a sole as disclosed herein.
Aspect 27: A method of making a shoe sole as disclosed herein.
Aspect 28: An insole made by a method as disclosed herein.
Aspect 29: A method to allow customization of insole stiffness based on plantar pressure as disclosed herein.
Aspect 30: A method to process patient specific patient specific data into lattice parameters to control regional insole stiffness.
Aspect 31: A method to generate lattice parameters throughout the insole.
Aspect 32: A method to derive and vary lattice parameters throughout the insole.

Although the foregoing invention has been described in some detail by way of illustration and example for purposes of clarity of understanding, certain changes and modifications may be practiced within the scope of the appended claims.

What is claimed is:

1. A method comprising:
generating, based on a profile of a foot and a patient-specific pressure distribution, an insole model.

2. The method of claim 1, wherein the profile of the foot comprises a scan of a crush box impression.

3. The method of claim 2, further comprising scanning the crush box impression.

4. The method of claim 1, further comprising forming an insole in accordance with the insole model.

5. The method of claim 1, wherein the profile of the foot is a 3-dimensional profile.

6. The method of claim 1, further comprising acquiring the patient-specific pressure distribution.

7. The method of claim 6, wherein the patient-specific pressure distribution comprises a reading from a pressure sensing pad.

8. The method of claim 1, wherein generating the insole model comprises generating a lattice.

9. The method of claim 8, wherein the lattice comprises a plurality of elongate segments and a plurality of intersections between elongate segments.

10. The method of claim 8, wherein the lattice comprises at least one repeating pattern.

11. The method of claim 10, wherein the at least one repeating pattern comprises hexagonal structures.

12. The method of claim 10, wherein the at least one repeating pattern comprises triangular structures.

13. The method of claim 8, wherein the lattice comprises at least one irregular shape.

14. The method of claim 1, further comprising manually editing the insole model.

15. The method of claim 14, wherein the lattice comprises a plurality of elongate segments and a plurality of intersections between elongate segments, wherein manually editing the insole model comprises changing a density of intersections and elongate segments.

16. The method of claim 14, wherein manually editing the insole model comprises changing a selected region of the insole model.

17. The method of claim 8, wherein the lattice comprises at least one lattice parameter.

18. The method of claim 17, wherein the lattice comprises a plurality of elongate segments and a plurality of intersections between elongate segments, wherein the at least one lattice parameter comprises one or more of: an overall material density, an elongate segment length, a density of elongate segments per unit volume, a density of intersections per unit volume, or an elongate segment thickness.

19. The method of claim 1, further comprising providing an interface to manually edit the insole model.

20. A method comprising:
forming, using additive manufacturing and an insole model based on a profile of a foot and a patient-specific pressure distribution, an insole.
